(19) United States
(12) Patent Application Publication
(10) Pub. No.: US 2009/0233313 A1
(43) Pub. Date: Sep. 17, 2009

(54) QUANTIFICATION AND AFFINITY CHARACTERIZATION OF ANTIBODIES FOR THE DIAGNOSIS OF DISEASE USING OPTICAL DIFFRACTION

(75) Inventors: Brian Pak, Maple (CA); Jean-Francois Houle, Toronto (CA); David Wong, Toronto (CA)

Correspondence Address: CLARK & ELBING LLP, 101 FEDERAL STREET, BOSTON, MA, 02110, US

(73) Assignee: Axela Inc., Toronto (CA)

(21) Appl. No.: 12/402,009

(22) Filed: Mar. 11, 2009

Related U.S. Application Data
(60) Provisional application No. 61/069,002, filed on Mar. 11, 2008.

(51) Int. Cl. G01N 33/53

(52) U.S. Cl. 435/7.1

(57) ABSTRACT

The invention features methods and devices for the detection of antibodies. The invention also features methods for diagnosing disease and evaluating the efficacy of treatment of a subject with a disease.

### CROSS-REFERENCE TO RELATED APPLICATIONS

This application claims benefit of U.S. Provisional Application No. 61/069,002, filed Mar. 11, 2008, which is hereby incorporated by reference.

### BACKGROUND OF THE INVENTION

In general, this invention relates to the fields of optical diffraction and biomarker detection.

Several antigens and antibodies have been identified as biomarkers for the diagnosis of disease. For example, blood levels of prostate specific antigen (PSA) have been used for many years as an indicator of the presence of prostate cancer. Antibodies directed against human tumor antigens may be promising sentinels in the early diagnosis of cancer, as the concentration of these antibodies is often much higher than the corresponding concentration of tumor antigens, making them easier to detect. Traditional methodologies for measuring the presence and concentration of antibodies present in biological samples, such as ELISA and Western blotting, often involve rigorous wash steps that may disrupt binding between an antigen and its antibody, particularly when affinity of the antibody for its antigen is low. In addition, these traditional methodologies provide no indication of the affinity between the antibody-antigen pair, which may be important when monitoring disease states in which antibody affinity changes with disease progression and treatment.

Thus, there exists a need in the art for methods to detect, quantify, and characterize in real time antibodies indicative of disease.

### SUMMARY OF THE INVENTION

The invention features methods and devices for the real-time detection of antibodies. The invention also features methods for diagnosing disease, evaluating the efficacy of treatment of a subject with a disease, and evaluating the affinity and/or avidity of an antibody bound to an antigen.

The invention features a device with a channel for liquid and having an immobilized antigen on a surface of the channel. The antigen specifically binds to an antibody expressed in response to the presence of a disease in a subject and is immobilized on the surface of the channel in a pattern that generates a signal, e.g., via diffraction. Binding of the antibody to the antigen causes a change in the signal generated by the pattern, e.g., the generation of signal compared to no signal or an increase in signal generated. The antibody of the invention does not otherwise bind to the surface of the device.

The invention also features a method of detecting an antibody that is expressed in response to the presence of a disease in a biological sample from a subject. The method includes contacting the biological sample with a device of the invention to allow any antibodies present to bind to the immobilized antigen. The signal produced by the extent of binding of the antibody is detected to determine the presence or absence of the antibody. This method may also be used to diagnose disease.

The method may also be used to evaluate the efficacy of treatment of a disease in a subject, wherein the disease results in the expression of an antibody in the subject. The method includes contacting a first biological sample from the subject, e.g., before treatment begins, and a second biological sample from the subject at a later time, e.g., after commencement of treatment, to a device of the invention to determine the amount of the antibody in the samples. A change in the amount of the antibody in the second sample compared to the first sample is indicative of the efficacy of treatment. Depending on the antibody detected, a decrease in amount may be indicative of successful treatment or of development of resistance. A change in affinity or avidity may alternatively be used to determine efficacy.

The invention further features a method of evaluating the affinity and/or avidity of an antibody bound to an antigen, e.g., wherein the antibody is expressed in response to the presence of a disease. The method includes contacting a biological sample from a subject with a device having an antigen that specifically binds to an antibody immobilized on a surface of the device in a pattern capable of generating a signal. Binding of the antibody to the antigen is then detected based on the signal generated to determine the presence or absence of the antibody. Affinity or avidity is determined based on the amount of antibody that binds in the presence of a solution or the amount of bound antibody that is eluted in the presence of a solution.

Any method of the invention may further include determining the concentration (relative or absolute) of the antibody in the biological sample, determining the rate of binding of the antibody to the antigen, determining the rate of dissociation of the antibody from the antigen, or determining the affinity or avidity of the antibody to the antigen, e.g., by determining the binding constant.

In certain embodiments of any aspect of the invention, the antibody expressed in response to the presence of a disease specifically binds to a compound administered to a subject to treat the disease. Monitoring the interaction between antibodies and compounds used in the treatment of a disease may be used to determine the presence or likelihood of the development of resistance to the treatment.

An exemplary disease that can be evaluated by the methods and devices of the present invention is cancer, e.g., prostate cancer, squamous cell cancer, small-cell lung cancer, non-small-cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, vulval cancer, thyroid cancer, hepatic carcinoma, gastric cancer, melanoma, or various types of head and neck cancer. Other diseases that may be evaluated by the methods and devices of the present invention are autoimmune diseases, e.g., autoimmune hepatitis, multiple sclerosis, systemic lupus erythematosus, myasthenia gravis, type I diabetes, rheumatoid arthritis, psoriasis, Hashimoto's thyroiditis, Graves' disease, Sjögren's syndrome, and scleroderma, or bacterial, viral, and fungal infections, e.g., hepatitis C or human immunodeficiency virus (HIV).

In one embodiment, the device includes PSA bound to the surface of the channel for the detection of anti-PSA antibodies. Additional antigens identified as biomarkers of disease that may be bound to the surface of the device are listed in Table 1.

The signal described in the methods and devices of the invention may be generated by the diffraction of light illuminating the device. The illumination may be by a laser.

The biological sample of the methods and devices of the invention is, e.g., blood, serum, plasma, cerebrospinal fluid, or urine.

Antibodies evaluated using the invention may be monoclonal antibodies (including full-length monoclonal antibodies), polyclonal antibodies, multispecific antibodies, or antigen-binding antibody fragments.

By “affinity” is meant a measure of the binding strength between one epitope and one paratope. Affinity can be measured by standard methods known in the art, including those described herein.

By “avidity” is meant a measure of the interaction between an antibody and its antigen. In contrast to the term “affinity,” avidity describes the strength of the interaction of antigen molecules with multiple epitopes with antibodies with more than one paratope.

By “antigen” is meant a molecule to which an antibody can selectively bind. The target antigen may be a polypeptide, carbohydrate, nucleic acid, lipid, hapten, or other naturally occurring or synthetic compound. Preferably, the target antigen is a polypeptide. An exemplary antigen is a tumor antigen (e.g., PSA). Other exemplary antigens are given in Table 1.

By “biological sample” is meant a sample obtained from a subject. Biological samples encompass, e.g., a clinical sample, cells in culture, cell supernatants, cell lysates, serum, plasma, biological fluid (e.g., urine), and tissue extracts. The source of the biological sample may be solid tissue (e.g., from a fresh, frozen, and/or preserved organ or tissue sample, biopsy, or aspirate), blood or any blood constituents, bodily fluids (such as, e.g., urine, cerebral spinal fluid, amniotic fluid, peritoneal fluid, or interstitial fluid), or cells from any time in gestation or development of the subject. In some embodiments, the biological sample is obtained from a primary or metastatic tumor. The biological sample may contain compounds that are not naturally intermixed with the tissue in nature such as preservatives, anticoagulants, buffers, fixatives, nutrients, or antibiotics.

By “cancer” is meant the physiological condition in mammals that is typically characterized by unregulated cell growth. Included in this definition are benign and malignant cancers, as well as dormant tumors or micro-metastases. Examples of cancer include, but are not limited to, carcinoma, lymphoma, blastoma, sarcoma, and leukemia. More particular examples of such cancers include, e.g., prostate cancer, squamous cell cancer, small-cell lung cancer, non-small-cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, vulval cancer, thyroid cancer, hepatic carcinoma, gastric cancer, melanoma, and various types of head and neck cancer.

By “detect” or “detection” is meant identification of the presence, absence, or amount of the substance or state to be detected.

By “immobilized” is meant bound directly or indirectly to a surface of, e.g., a device, including attachment by covalent binding or non-covalent binding (e.g., hydrogen bonding, ionic interactions, or hydrophobic interactions).

By “signal” is meant light (e.g., light generated by fluorescence, bioluminescence, or phosphorescence), ionizing radiation, particle emission, magnetism, staining, or a product of a reaction involving an enzyme. Diffraction, absorbance, polarization, reflection, deflection, increases, decreases, or amplification of a signal may be indicative of an event (e.g., binding of an antibody to an antigen immobilized on the surface of a diffraction-based device).

An antibody that “specifically binds” is an antibody or fragment thereof that recognizes and binds an antigen, but that does not substantially recognize or bind to other molecules in a biological sample. Specific recognition of an antigen by an antibody may be assayed by using, e.g., light diffraction devices with an immobilized capture surface or using standard techniques known to one of skill in the art, such as immunoprecipitation, Western blotting, and ELISA.

By “subject” is meant humans and other animals including, e.g., mice, rats, guinea pigs, hamsters, rabbits, cats, dogs, goats, sheep, cows, or monkeys.

Other features and advantages of the invention will be apparent from the following description, drawings, and claims.

### BRIEF DESCRIPTION OF THE DRAWINGS

FIG. 1A is a graph showing typical results of prostate-specific antigen (PSA) antibody binding to immobilized PSA on the surface of a device after blocking and washing steps. FIG. 1B is a graph showing binding of anti-PSA antibody in plasma (top curve) to the surface of the device compared to control plasma (bottom curve). FIG. 1C is a representation of biotinylated PSA molecules immobilized on the surface of the device through an interaction with avidin molecules bound to the device in a pattern that produces a diffraction pattern when illuminated with light.

FIG. 2 is a graph showing the binding of plasma samples to a PSA-conjugated device. The plasma samples were taken from three prostate cancer patients (PC+) and two subjects that have not been diagnosed with prostate cancer (PC−).

FIG. 3 is a graph comparing the results of a digital rectal examination (DRE) with the binding assay of the present invention. The results of the DRE were compared to the results of the binding assay at the 30- and 60-second time points (e.g., time points at which an increase in the diffractive signal is observed). The graph shows that two of the subjects with prostate cancer (e.g., 4+3 and 4+4) had normal DRE results. The two subjects who had not been diagnosed with prostate cancer had abnormal DRE results. The binding assay described herein correctly classified each subject.

FIG. 4 is a graph showing typical results of an antibody dissociation assay. Following antibody binding, dissociation was induced by washing the device with either dissociation buffer (e.g., BSA-PBST) or free PSA in BSA-PBST as a competitor for binding to the antibody. The graph shows that dissociation buffer alone (light gray curve) does not cause significant antibody dissociation, whereas free PSA results in significant antibody dissociation (gray and black curves).

FIG. 5 is a graph showing the amplification of a PSA autoantibody-specific binding curve with 18-nm gold colloid nanoparticles. “g α h” is goat anti-human PSA antibody and “d α g” is donkey anti-goat IgG antibody.

### DETAILED DESCRIPTION OF THE INVENTION

The invention features methods and devices for the detection of antibodies, e.g., for diagnosing disease and evaluating the efficacy of treatment. The methods of the invention include contacting a biological sample with a device having an antigen on its surface. The antigen binds to an antibody present in the sample, forming a complex on the surface of the device. The signal may be detected using detection methods known to those skilled in the art (e.g., optical diffraction).

### Antibodies and Antigens

Different antibodies may be detected by methods described herein, e.g., an antibody produced in the body in response to a tumor antigen. The antibodies may be present in a biological sample (e.g., blood, serum, plasma, crude cell lysates, or urine). The biological sample obtained from the subject may contain various antibody clones (e.g., polyclonal antibodies).

Various concentrations of antibodies may be detected and measured by the methods described herein. Antibodies present at concentrations less than, e.g., 100 milligrams/milliliter (mg/ml), 10 mg/ml, 1 mg/ml, 100 micrograms/milliliter (μg/ml), 10 μg/ml, 1 μg/ml, 100 nanograms/milliliter (ng/ml), 10 ng/ml, 1 ng/ml, 100 picograms/milliliter (pg/ml), 10 pg/ml, 1 pg/ml, 100 femtograms/milliliter (fg/ml), or 10 fg/ml may be detected in the biological sample, and the concentration may be measured.

The devices described in the methods and compositions of the invention described herein contain antigens immobilized on the surface of the device. The antigen may include any substance capable of binding an antibody. Antibodies may bind covalently or non-covalently to the antigen. The antigen may be a tumor antigen (e.g., PSA) that specifically binds to an antibody (e.g., anti-PSA antibody). Other tumor-associated antigens that may be immobilized on the surface of the device include, e.g., tyrosinase, MUC1, p53, CEA, pmel/gp100, ErbB-2, MAGE-A1, NY-ESO-1, and TRP-2 (see, e.g., U.S. Pat. Nos. 5,102,663; 5,141,742; 5,262,177; 5,538,866; 4,816,249; 5,068,177; and 5,227,159, hereby incorporated by reference). Additional antigens identified as biomarkers of disease are listed in Table 1 and are described, e.g., in U.S. Pat. Nos. 4,468,465; 5,856,112; 6,251,613; 6,280,941; 6,699,675; 6,753,135; 7,001,775; 7,037,651; 7,144,569; 7,189,516; and 7,262,062, hereby incorporated by reference. The antigen may also be a therapeutic agent (or hapten thereof) used to treat a disease.

TABLE 1

| Antigen Description | Clinical Association |
|---|---|
| Prostate specific antigen (PSA) | Prostate cancer and other prostate disorders |
| Tyrosinase | Melanoma and breast cancer |
| MUC1 | Breast cancer; lung cancer |
| p53 | Cervical cancer; Li-Fraumeni syndrome; |
| | malignant human tumors |
| Carcinoembryonic antigen (CEA) | Intestinal cancers; pancreatic cancer; breast cancer; |
| | lung cancer; pancreatitis; cirrhosis; benign tumors |
| pmel/gp100 | Melanoma |
| ErbB-2 (HER2/neu) | Breast cancer; ovarian cancer; stomach cancer; |
| | endometrial carcinoma |
| MAGE-A1 | Melanoma; colorectal cancer; liver cancer; lung |
| | cancer; and hematologic neoplasms |
| NY-ESO-1 | Multiple myeloma; breast cancer |
| TRP-2 | Melanoma |
| Thyroid peroxidase (TPO) | Graves' disease |
| Thyroglobulin (TG) | Hashimoto thyroiditis |
| dsDNA; proliferating cell nuclear antigen (PCNA) | Systemic lupus erythematosus (SLE) |
| Ro/SS-A; La/SS-B | Subacute cutaneous lupus erythematosus; neonatal |
| | lupus; primary and secondary Sjögren's syndrome |
| Ribosomal phosphoprotein P0 | SLE (CNS involvement) |
| U1-snRNP 68 protein (68 kDa); U1-snRNP A | Mixed connective tissue disease; SLE |
| protein; U1-snRNP C protein; U-snRNP protein | |
| B/B′ | |
| Histidyl-tRNA synthetase (Jo-1); threonyl-tRNA | Polymyositis; dermatomyositis |
| synthetase (PL-7); alanyl-tRNA synthetase (PL- | |
| 12); SRP54 | |
| Mi-2 | Dermatomyositis |
| PM/Scl 75 (BV); PM/Scl 100 (BV) | Polymyositis; scleroderma |
| DNA topoisomerase I (Scl-70) | Scleroderma (diffuse) |
| Centromere protein A (CENP-A); centromere | Scleroderma (CREST) |
| protein B (CENP-B) | |
| Ku (p70/p80) | Overlap syndrome |
| Cytochrome P450 2D6 (LKM 1); | Autoimmune hepatitis II |
| formiminotransferase cyclodeaminase (LC1) | |
| M2 | Primary biliary cirrhosis |
| β2 glycoprotein I (apolipoprotein H) | Thromboembolic syndrome |
| Glomerular basement membrane (undissociated); | Goodpasture's syndrome |
| glomerular basement membrane (dissociated) | |
| Gliadin; tissue transglutaminase | Celiac disease |
| Intrinsic factor | Pernicious anemia |
| Myeloperoxidase (MPO) | ANCA associated diseases |
| Islet cell antigen (ICA); insulin; glutamic acid | Type I diabetes |
| decarboxylase (GAD 65); IA-2 | |
| Myelin basic protein (MBP) | Multiple sclerosis |

The antigen immobilized on the device will ultimately depend on the antibody being assayed. The antigen may be bound to the device by methods known to one of skill in the art, such as a biotin-avidin or biotin-streptavidin interaction, a Protein A interaction, a Protein G interaction, a GAM-Fc interaction, an amide bond, or through any other covalent or non-covalent interaction.

### Methods to Detect and Measure an Antibody in a Biological Sample

The signal produced upon the binding of an antibody to the device of the invention described herein may be detected or measured using any technique known in the art, including optical diffraction. Exemplary techniques for detection are provided in, e.g., U.S. Pat. No. 6,991,938, hereby incorporated by reference.

Methods for using optical diffraction-based assays will be known to those skilled in the art and are described in, e.g., U.S. Pat. Nos. 7,008,794 and 7,314,749, U.S. Patent Application Publication No. 2006/0099649, and in Goh et al. (“Diffraction-Based Assay for Detecting Multiple Analytes,” Anal. Bioanal. Chem. 374: 54-56, 2002), which are hereby incorporated by reference.

Diffraction-based assays involve immobilizing an antigen in a distinct pattern on the surface of a device. In one embodiment, the antigens are immobilized in distinct locations or assay spots (e.g., up to eight distinct locations or assay spots) on the surface of a device in a pattern (e.g., a series of parallel lines) that produces a diffraction pattern when illuminated with light (e.g., light with a wavelength in the range from the ultraviolet to the infrared, but preferably a coherent and collimated light beam, such as would come from a laser (e.g. diode, He—Ne, Nd:YVO₄, or Argon-ion laser)) (see, e.g., U.S. Patent Application Publication No. 2006/0099649).

Once the antigen is immobilized on the device, the biological sample to be assayed is contacted with the device (e.g., by flowing the sample through the device), allowing antibodies present in the sample to bind to the antigen on the surface of the device. When a particular antibody is present in the biological sample being tested, the subsequent binding event between the antibody and antigen is accompanied by a change in the local thickness of the surface of the device and/or in the local index of refraction. Both the change in thickness and the change in refractive index will alter the optical properties at the interface between the device and sample in regions where binding has taken place. Since the antigens are present on the device in a predetermined pattern, light incident on the surface of the device will not be scattered uniformly but rather will be diffracted. In one embodiment of this invention, the patterned substrate is non-diffracting, and the binding events result in an observable diffraction image. Alternatively, the patterned surface of the device itself produces an observable diffraction image, but the binding events alter the intensities of the diffracted signal. The intensity of the diffraction signal may be used to generate real-time binding curves. In one embodiment, the illumination and detection beams never pass through the sample, which is particularly advantageous for the detection of proteins in complex biological samples. See, e.g., U.S. Pat. No. 7,314,749, hereby incorporated by reference.

Since the diffraction-based detection of binding events is dependent on the pattern of the immobilized antigens, a change in signal occurs only when antibodies bind exclusively to the immobilized antigens. Non-specific binding to the surface of the devices employed by the invention generally produces little or no change in the diffraction signal. This label-free characteristic of the invention enables the direct study of multiple biomolecular interactions in parallel, including, e.g., protein-protein interactions. The optical diffraction signals of antibodies being measured may be measured directly (measuring direct binding without amplification by additional moieties) or indirectly by using additional moieties to amplify the signal such as, e.g., horseradish peroxidase, a bead, nanoparticles, or alkaline phosphatase.

Detection of the diffraction signal depends on the source of illumination. The detector may be, e.g., a position sensitive photodiode, a photomultiplier tube (PMT), a photodiode (PD), an avalanche photodiode (APD), a charged-coupled device (CCD) array, the unaided eye, a camera, a photographic plate, or any other imaging device. The detector may be attached to the appropriate accessories to provide power and enable signal collection and data processing.

The device used in a diffraction-based assay is typically a flow-through device having a channel for fluid to contact the patterned antigen. The patterns on the surface of the device may be created using microlithography, microcontact printing, inkjet writing, robotic spotting, dip pen nanolithography, nanolithography by atomic force microscopy, or near-field optical scanning lithography. The device may be made of any suitable material (e.g., a synthetic polymer (e.g., polystyrene), glass, metal, silicon, or semiconductor). Depending on the choice of material, the device employed may be disposable. An exemplary device is described in U.S. Pat. No. 7,314,749, hereby incorporated by reference.

The surface of the device may be coated with different immobilized binding agents known in the art. Immobilized avidin groups on the surface of the device may be used for high-affinity immobilization of biotinylated binding agents (e.g., biotinylated antigens). For example, a biotinylated antigen that specifically binds to an antibody may be immobilized on the surface of an avidin-coated device. Protein G on the surface of the device may bind to the Fc region of immunoglobulin molecules, allowing oriented immobilization of antibodies as binding agents on the surface of the device. Goat anti-mouse-Fc (GAM-Fc)-coated surfaces bind to the Fc region of mouse antibodies, allowing oriented immobilization of mouse antibodies on the surface of the device employed by the invention.

Immobilized carboxylate groups on an amine-reactive surface may be used to covalently link binding agents (e.g., with amide bonds) to the device's surface via an amine-coupling reaction. Other exemplary reactive linking groups, e.g., hydrazines, hydroxylamines, thiols, carboxylic acids, epoxides, trialkoxysilanes, dialkoxysilanes, and chlorosilanes may be attached to the surface of the device, such that binding agents may form chemical bonds with those linking groups to immobilize them on the surface of the device. Appropriate devices are commercially available from Axela, Inc. (Toronto, Canada).

### Uses of the Invention

The invention described herein features methods for diagnosing disease and evaluating the efficacy of treatment of a subject with a disease. Physicians and researchers may use the methods of the invention to detect antibodies (e.g., antibodies against tumor antigens) or may use the methods of the invention to diagnose or screen for disease (e.g., cancer or autoimmune diseases).

Diagnosis of Disease

The methods described herein may be used to diagnose a disease (e.g., cancer, an autoimmune disease, or an infection) in a subject. For example, the methods of the invention may be used to diagnose a disease in a subject that results in the expression of an antibody. A diagnosis may be made if, for example, the presence of the antibody is detected in a biological sample obtained from the subject.

The disease being diagnosed may be cancer (e.g., a carcinoma, lymphoma, blastoma, sarcoma, or leukemia). More particular examples of such cancers include, e.g., prostate cancer, squamous cell cancer, small-cell lung cancer, non-small-cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, vulval cancer, thyroid cancer, hepatic carcinoma, gastric cancer, melanoma, and various types of head and neck cancer. The disease may also be an autoimmune disease, e.g., autoimmune hepatitis, multiple sclerosis, systemic lupus erythematosus, myasthenia gravis, type I diabetes, rheumatoid arthritis, psoriasis, Hashimoto's thyroiditis, Graves' disease, Sjögren's syndrome, or scleroderma.

The methods described herein may also be used to diagnose infections (e.g., bacterial or viral infections). Exemplary bacteria, viruses, and fungi that may lead to an infection include hepatitis C, human immunodeficiency virus (HIV), adenovirus type 2 hexon, Aspergillus fumigatus, Borrelia afzelii, Borrelia gaminii, Campylobacter jejuni, Candida albicans, Chlamydia, coxsackievirus B1, coxsackievirus B5, coxsackievirus B6, cytomegalovirus, Echinococcus, echovirus type 6, Helicobacter pylori, Herpes simplex virus types 1 and 2, HTLV-1, human papillomavirus, hepatitis B, influenza A virus, influenza B virus, Legionella pneumophila, Leptospira biflexa, measles virus, mumps virus, Mycoplasma pneumoniae, parainfluenza virus types 1, 2, and 3, respiratory syncytial virus, rubella virus, Toxoplasma gondii, and Varicella-Zoster virus.

Evaluating the Efficacy of Treatment

The methods described herein may be used to evaluate the efficacy of treatment of a disease of a subject. Such an evaluation includes, e.g., obtaining at least one biological sample from the subject typically before treatment begins, as well as obtaining at least one biological sample from the subject any time after commencement of the treatment (e.g., 1, 2, 3, 4, 5, or 6 days; 1, 2, or 3 weeks; 1, 2, 3, 4, 5, 6, 7, 8, 9, 10, or 11 months; or 1, 2, 3, 4, or 5 years after treatment has begun). The pre- and post-treatment samples may then be applied to a device containing an immobilized antigen that is capable of specifically binding to an antibody that is indicative of the disease. The efficacy of treatment may then be evaluated by comparing the amount of antibody in each sample. For example, a decrease in the amount of the antibody in the sample obtained after treatment commenced may be an indication that the treatment of the disease is efficacious. The presence of antibodies produced in a subject during treatment of a disease may also be determined using the methods described herein, e.g., to determine the onset or extent of resistance to treatment.

These methods may be used in the absence of treatment to determine disease prognosis, progression, or natural healing.

Evaluating the Affinity and/or Avidity of an Antibody Bound to an Antigen

The methods described herein may be used to evaluate the affinity and/or avidity of an antibody bound to an antigen in a biological sample. The affinity and/or avidity of the binding between the antibody-antigen pair may be used to diagnose disease or to determine the stage of the disease or the length of the disease. Unlike the present methods, tandard immunoassays may only recognize binding interactions between high affinity/avidity antibody-antigen complexes present at high concentrations because of the harsh wash protocols employed. The affinity/avidity between an antibody-antigen pair may change (e.g., increase) as a subject is subjected to repeated and/or increasing doses of antigen (e.g., during tumor growth). For example, as a tumor grows, antibodies are produced and may mature (both in concentration and avidity) in parallel with tumor growth, ultimately being diagnostic of tumor growth and useful in monitoring therapeutic treatment and relapse.

In certain embodiments of the present invention, the affinity and/or avidity of an antibody for an antigen may be determined by contacting a biological sample from a subject with a device having an antigen that specifically binds to an antibody immobilized on a surface thereof in a pattern capable of generating a signal so that the antibody binds to the antigen. Binding of the antibody to the antigen is then detected based on the signal generated to determine the presence or absence of the antibody. The surface of the device is then washed with a solution, and the signal is evaluated to determine a change in the amount of bound antibody to determine the affinity and/or avidity of the antibody-antigen bond. The wash solution may contain free antigen that specifically binds to the antibody.

Affinity and/or avidity may be measured with the device of the present invention using competitive inhibition assays or elution assays, such as those described in Pullen et al. (J Immunol Methods 86: 83-87 (1986)) or McCloskey et al. (J Immunol Methods 205: 67-72 (1997)), hereby incorporated by reference. For example, in competitive inhibition assays, wash solution containing free antigen is added to a device with antigen immobilized on its surface, and the amount of free antigen which inhibits antibody binding to the immobilized antigen by, e.g., 50% is determined. The less free antigen needed to inhibit antibody binding to the immobilized antigen, the stronger the affinity and/or avidity of the antigen-antibody interaction. In elution assays, a chaotrope or denaturant agent (e.g., isothiocyanate, urea, or diethylamine) is added to the device to disrupt antibody/antigen interactions, and the amount of antibody resisting elution is determined to measure the affinity and/or avidity.

The methods of the invention may also speed the detection of an antibody in a number of ways, including, e.g., quantifying antibody concentration and purity, characterizing binding kinetics, determining specificity and cross-reactivity, optimizing antibody concentrations (relative or absolute), step times, buffers, and additive composition, monitoring assay performance and matrix effects, and multiplexing antibodies with minimized interference.

### EXAMPLES

### Example 1

### Detection and Characterization of Prostate-Specific Antigen (PSA) Autoantibody

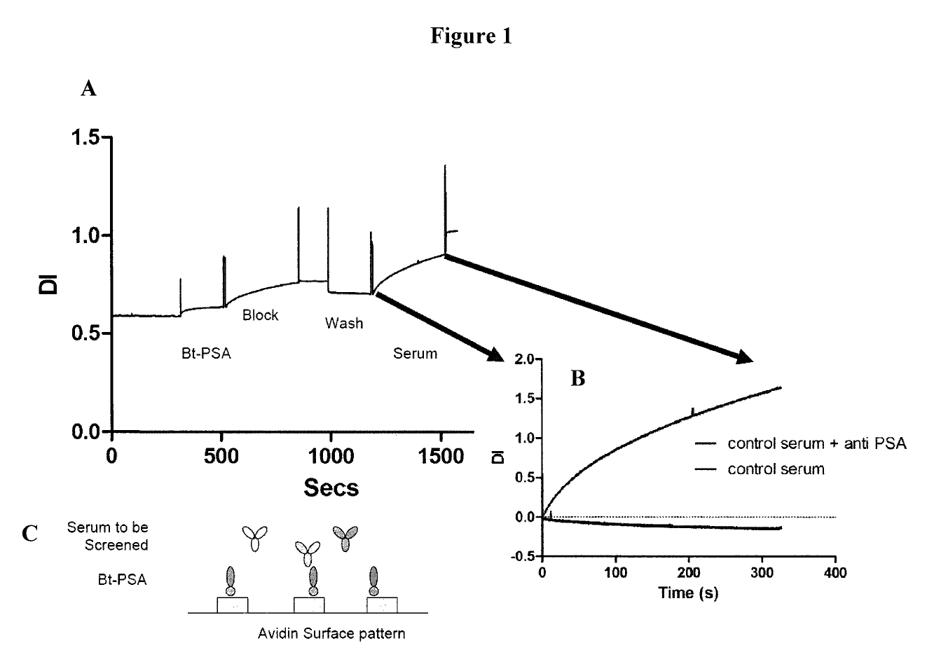

An avidin diffraction sensor device (a DotLab™ avidin device, Axela Inc., Canada) was first blocked with 5 mg/ml of bovine serum albumin (BSA) in a solution of phosphate-buffered saline (PBS) with 0.05% Tween-20 (BSA-PBST). The device was then conjugated with 1.5 μg/ml of biotin-tagged PSA (Fitzgerald Industries International, USA). To control for non-specific binding of plasma proteins, the PSA-coated devices were incubated 3-4 minutes with plasma samples (1:10 dilution of plasma in BSA-PBST) from subjects that had not been diagnosed with prostate cancer. Following this blocking step, the devices were then incubated with plasma samples (1:10 dilution in BSA-PBST) from subjects diagnosed with prostate cancer or control plasma (1:10 dilution in BSA-PBST) to determine PSA autoantibody levels in the plasma sample or control sample (FIG. 1). Autoantibody binding to the device was determined by monitoring the intensity of the diffraction signal upon introduction of the samples to the device. Following autoantibody binding, autoantibody affinity was determined by monitoring dissociation of the autoantibody from the PSA-conjugated surface of the device. Dissociation was induced by washing the devices with either dissociation buffer (BSA-PBST) or 15.6 μg/ml of free PSA in BSA-PBST as a competitor for autoantibody binding and incubating the devices in the solutions for 1 hour.

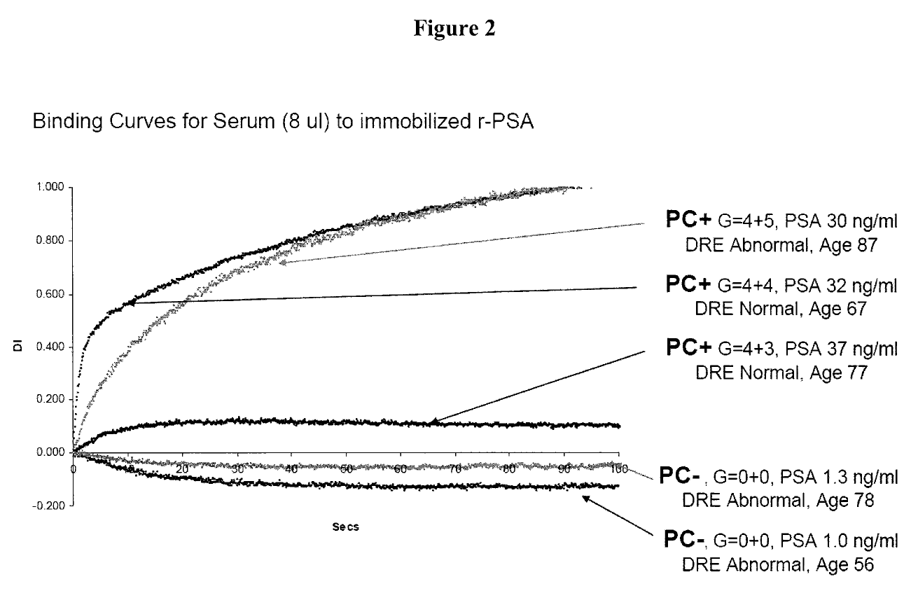

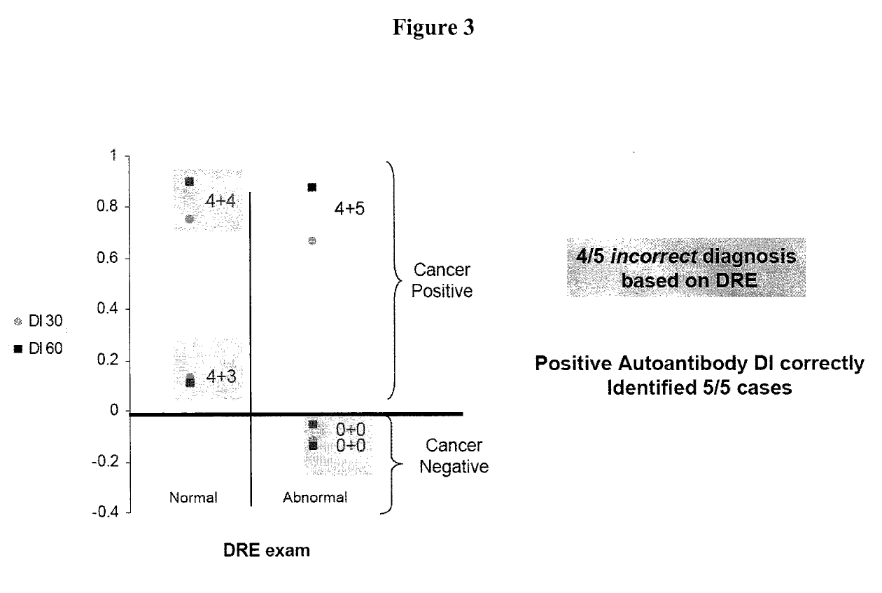

Typical results demonstrated that plasma from subjects diagnosed with prostate cancer (control plasma+anti-PSA; top curve of FIG. 1) showed a significant autoantibody binding signal (e.g., an increase in diffraction intensity over time) while no signal was observed with the control plasma sample from a subject not diagnosed with prostate cancer (control plasma; bottom curve of FIG. 1). These results were also observed when plasma samples from three subjects diagnosed with prostate cancer (PC+; FIG. 2) were tested and compared to plasma samples from two subjects not diagnosed with prostate cancer (PC−; FIG. 2). All plasma samples derived from subjects with prostate cancer showed an autoantibody binding signal while no signal was observed from the control samples. The results of these assays were compared to the results of digital rectal examinations (DRE) performed on each subject to screen for prostate cancer. This comparison showed that 4 out of 5 of the DRE diagnoses were incorrect (FIG. 3). In this sampling, two subjects with prostate cancer had a normal DRE, and their cancer would have not have been detected. Two patients with an abnormal DRE were cancer-free. In contrast, the measurement of autoantibody binding to the device correctly classified all subjects (FIG. 3).

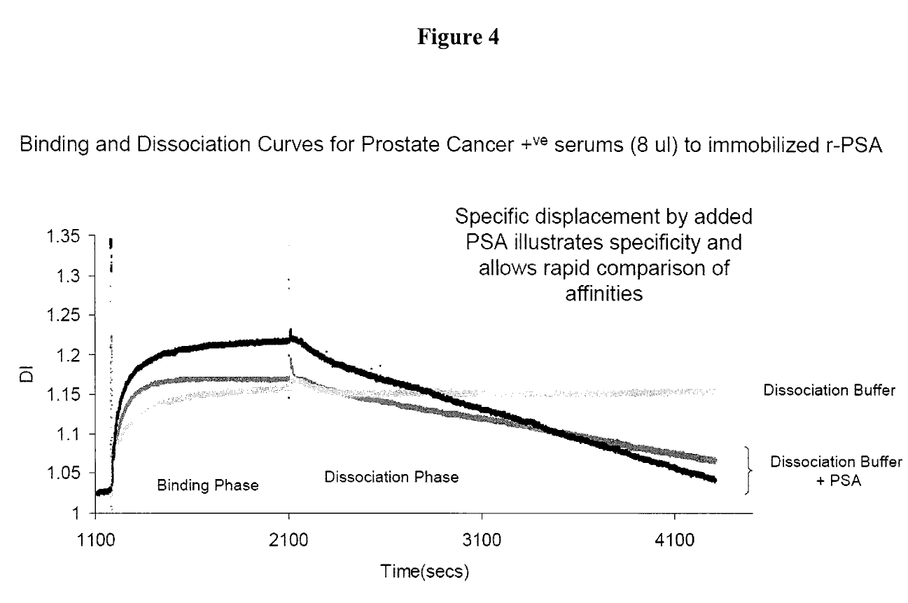

Results from an autoantibody dissociation assay demonstrated that dissociation buffer alone (e.g., in the absence of free PSA) does not cause significant autoantibody dissociation (FIG. 4). However, significant autoantibody dissociation was observed using dissociation buffer in the presence of free PSA (FIG. 4).

### Example 2

### Enhanced Autoantibody Detection Using Gold Colloid Nanoparticles

All assays were performed on an avidin diffraction sensor device (a DotLab™ avidin device, Axela Inc., Canada) using a running buffer of PBS with 0.05% Tween-20 (PBST). Blocking was performed using a 1:5 dilution of normal human serum in PBST plus 5 mg/ml bovine serum albumin (PBST-BSA). All washes were performed at a flow rate of 500 μl/min, and all sample/reagent incubations were performed at a flow rate of 500 μl/min. Real-time visualization of autoantibodies was achieved by the addition of a 1:20 dilution of 18-nm gold colloid nanoparticles diluted in PBST-BSA.

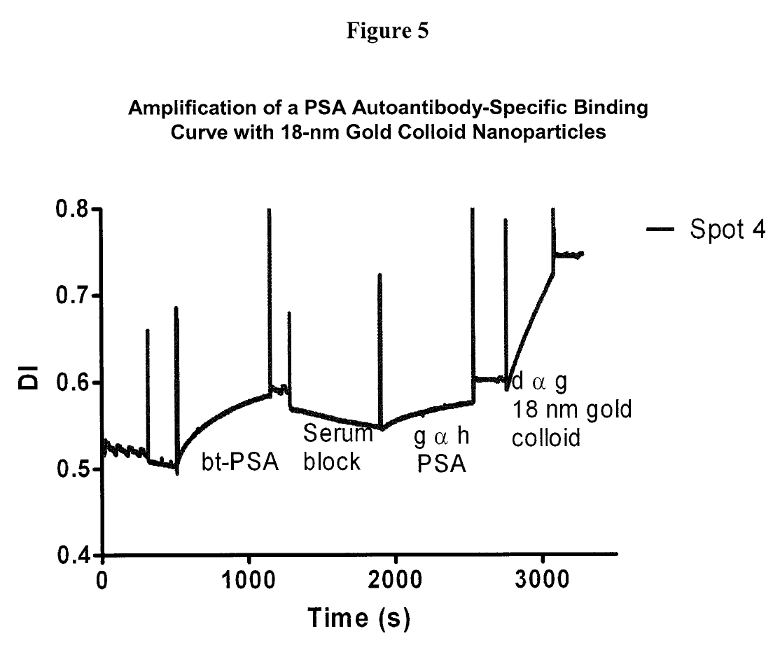

Following a series of washes with the running buffer to wet the sensor surface and a brief blocking step with PBST-BSA to eliminate non-specific binding to the sensor surface, 1.5 μg/ml of biotinylated human PSA (bt-PSA) protein was applied to the sensor and incubated for 5 minutes. The sensor was washed briefly with running buffer followed by a second blocking step with a 1:5 dilution of normal human serum in PBST-BSA. The serum sample of interest containing the goat anti-human PSA autoantibody (g α h PSA, FIG. 5) was then incubated for 5 minutes with the sensor. The sensor was then washed briefly with PBST. Colloidal Gold-AffiniPure Donkey Anti-Goat IgG 18-nm nanoparticles (Jackson ImmunoResearch Laboratories, Inc., West Grove, Pa.; Catalog No.: 705-215-147) were diluted in PBST-BSA (1:20 dilution) and incubated for 5 minutes with the sensor to amplify the PSA autoantibody signal (d α g 18 nm gold colloid; FIG. 5). Upon amplification with the antibody-conjugated gold colloid nanoparticles, a PSA autoantibody-specific binding curve was observed (FIG. 5). The magnitude of the nanoparticle signal is directly proportional to the quantity of autoantibody present in the serum sample.

### OTHER EMBODIMENTS

All publications, patents, and patent applications mentioned in the above specification are hereby incorporated by reference. Various modifications and variations of the described method and system of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Other embodiments are in the claims.

1. A method of detecting an antibody in a biological sample from a subject, said antibody expressed in response to a disease in said subject, said method comprising:
(a) contacting said biological sample with a device comprising an antigen that is immobilized on a surface thereof in a pattern that generates a signal upon binding of said antibody and that specifically binds to said antibody, and allowing said antibody in said sample to bind to said antigen; and
(b) detecting binding of said antibody to said antigen based on said signal to determine the presence or absence of said antibody.

2. The method of claim 1, further comprising determining the concentration of said antibody in said biological sample, determining the rate of binding of said antibody to said antigen, or determining a binding constant for said antibody to said antigen.

3. The method of claim 1, wherein said disease is cancer.

4. The method of claim 3, wherein said cancer is prostate cancer, squamous cell cancer, small-cell lung cancer, non-small-cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, vulval cancer, thyroid cancer, hepatic carcinoma, gastric cancer, melanoma, or various types of head and neck cancer.

5. The method of claim 1, wherein said disease is an autoimmune disease.

6. The method of claim 5, wherein said autoimmune disease is autoimmune hepatitis, multiple sclerosis, systemic lupus erythematosus, myasthenia gravis, type I diabetes, rheumatoid arthritis, psoriasis, Hashimoto's thyroiditis, Graves' disease, Sjögren's syndrome, or scleroderma.

7. The method of claim 1, wherein said antibody is anti-prostate-specific antigen (PSA) antibody and said antigen is PSA.

8. The method of claim 1, wherein said signal is generated by diffraction of light illuminating said device.

9. The method of claim 1, further comprising diagnosing said disease in said subject based on the presence or absence of said antibody.

10. A method of evaluating the efficacy of treatment of a disease in a subject expressing an antibody in response to said disease or treatment thereof, said method comprising:
(a) contacting a first biological sample with a first device comprising an antigen that specifically binds to said antibody and that is immobilized on a surface of said first device in a pattern that generates a first signal, and allowing said antibody in said first sample to bind to said first device;
(b) contacting a second biological sample with a second device comprising said antigen that specifically binds to said antibody and that is immobilized on a surface of said device in a pattern that generates a second signal, and allowing said antibody in said second sample to bind to said second device;
(c) detecting the amount of said antibody in said first and second samples based on said first and second signals signal; and
(d) comparing said amount of said antibody in said first and second samples, wherein a difference in the amount of said antibody in said second sample is indicative of the efficacy of treatment.

11. The method of claim 10, wherein said disease is cancer.

12. The method of claim 11, wherein said cancer is prostate cancer, squamous cell cancer, small-cell lung cancer, non-small-cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, vulval cancer, thyroid cancer, hepatic carcinoma, gastric cancer, melanoma, or various types of head and neck cancer.

13. The method of claim 10, wherein said disease is an autoimmune disease.

14. The method of claim 13, wherein said autoimmune disease is autoimmune hepatitis, multiple sclerosis, systemic lupus erythematosus, myasthenia gravis, type I diabetes, rheumatoid arthritis, psoriasis, Hashimoto's thyroiditis, Graves' disease, Sjögren's syndrome, or scleroderma.

15. The method of claim 10, wherein said antibody is anti-PSA antibody and said antigen is PSA.

16. The method of claim 10, wherein said signal is generated by diffraction of light illuminating said device.

17. A device comprising a channel for liquid and comprising an antigen selected from any one of the antigens of Table 1, wherein said antigen is immobilized on a surface of said channel in a pattern that generates a signal when an antibody specifically binds to said antigen.

18. The device of claim 17, wherein said antibody is anti-PSA antibody and said antigen is PSA.

19. The device of claim 17, wherein said signal is generated by diffraction of light illuminating said device.

20. A method of evaluating the affinity and/or avidity of an antibody for an antigen, said antibody expressed in response to the presence of a disease in a subject, said method comprising:
(a) contacting a biological sample from said subject with a device comprising said antigen that is immobilized on a surface thereof in a pattern capable of generating a signal and that specifically binds to said antibody, and allowing said antibody in said sample to bind to said antigen;
(b) detecting binding of said antibody to said antigen based on said signal; and
(c) determining the affinity and/or avidity of the antibody-antigen bond from a change in the amount of antibody bound to said antigen, wherein either (i) the binding in step (a) occurs in the presence of a substance that inhibits binding of said antibody or (ii) after step (b) said surface of said device is washed with a solution to dissociate said antibody from said antigen.

21. The method of claim 20, further comprising determining the rate of dissociation of said antibody from said antigen.

22. The method of claim 20, wherein said disease is cancer.

23. The method of claim 22, wherein said cancer is prostate cancer, squamous cell cancer, small-cell lung cancer, non-small-cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, vulval cancer, thyroid cancer, hepatic carcinoma, gastric cancer, melanoma, or various types of head and neck cancer.

24. The method of claim 20, wherein said disease is an autoimmune disease.

25. The method of claim 24, wherein said autoimmune disease is autoimmune hepatitis, multiple sclerosis, systemic lupus erythematosus, myasthenia gravis, type I diabetes, rheumatoid arthritis, psoriasis, Hashimoto's thyroiditis, Graves' disease, Sjögren's syndrome, or scleroderma.

26. The method of claim 20, wherein said antibody is anti-PSA antibody and said antigen is PSA.

27. The method of claim 20, wherein said signal is generated by diffraction of light illuminating said device.

28. The method of claim 20, wherein said solution comprises free antigen that specifically binds to said antibody.
